(19) United States
(12) Patent Application Publication
(10) Pub. No.: US 2021/0229887 A1
(43) Pub. Date: Jul. 29, 2021

(54) FOLDABLE CARD AND PACKAGE FOR SURGICAL INSTRUMENTS

(71) Applicant: Covidien LP, Mansfield, MA (US)

(72) Inventors: Nolan A. Hughes, Branford, CT (US); Alexander Y. Ueno, Hamden, CT (US); Douglas M. Pattison, East Hartford, CT (US); Christopher A. Tokarz, Torrington, CT (US); Kevin Desjardin, Cheshire, CT (US); Oksana Buyda, East Haven, CT (US); Amanda M. Adinolfi, Wallingford, CT (US); Astley C. Lobo, West Haven, CT (US)

(21) Appl. No.: 16/751,518

(22) Filed: Jan. 24, 2020

(51) Int. Cl. B65D 77/26; B65D 77/00; A61B 50/30

(52) U.S. Cl. CPC B65D 77/26; A61B 2050/314; A61B 50/30; B65D 77/003

(57) ABSTRACT

A foldable card for retaining a plurality of surgical instruments is disclosed. The foldable card includes a first panel, a second panel hingedly connected to the first panel, a third panel hingedly connected to the second panel, a fourth panel hingedly connected to the third panel, a fifth panel hingedly connected to the fourth panel, and a sixth panel hingedly connected to the fifth panel. The first panel defines a first opening configured to allow a portion of a first surgical instrument to pass therethrough, and a second opening configured to allow a portion of a second surgical instrument to pass therethrough. The third panel defines a first opening configured to allow a portion of the first surgical instrument to pass therethrough, and a second opening configured to allow a portion of the second surgical instrument to pass therethrough.

### TECHNICAL FIELD

The present disclosure relates to packaging products for surgical instruments. More particularly, the present disclosure relates to a foldable card and package for surgical instruments.

### BACKGROUND

Wrap-around storage containers are well-known. When fabricating a storage container from a paperboard blank, opposite sides of the blank are often attached to each other by glue or by mechanical locks to form the bottom panel of the storage container. In the case of a wrap-around storage container, flaps located on the sides of the blank typically are overlapped and engaged with one another by mechanical locks formed in the flaps to form the bottom panel of the storage container.

Surgical instruments have been known to be packaged in a similar configuration called a “blister.” Blister packages, which are also called “blister packs,” are known in a wide variety of embodiments. In the previously known blister packages, a product which is also intended as an insert or accessory part is usually packaged visibly. To this end, the previously known blister packages have a plastic film molded part which is manufactured from transparent plastic film and supports the product to be packaged in a molded cavity which is made in the plastic film. Blister packages of this type allow the customer, purchaser, or user to see the packaged article. The product is presented in front of a cardboard wall and is fixed with the plastic film molded part. The plastic film may also act as a sterile barrier between the surgical instrument within the package, and the ambient environment.

Certain surgical procedures require using several types of surgical instruments. Accordingly, it may be helpful to provide product packaging that is configured to house several surgical instruments. Additionally, it would be helpful for the product packaging to minimize or prevent the surgical instruments from unintended movement during shipping and handling, which would also help prevent damage to the packaging itself including any sterile barrier included with the packaging.

### SUMMARY

The present disclosure relates to a foldable card for retaining a plurality of surgical instruments. The foldable card includes a first panel, a second panel hingedly connected to the first panel, a third panel hingedly connected to the second panel, a fourth panel hingedly connected to the third panel, a fifth panel hingedly connected to the fourth panel, and a sixth panel hingedly connected to the fifth panel. The first panel defines a first opening configured to allow a portion of a first surgical instrument to pass therethrough, and a second opening configured to allow a portion of a second surgical instrument to pass therethrough. The third panel defines a first opening configured to allow a portion of the first surgical instrument to pass therethrough, and a second opening configured to allow a portion of the second surgical instrument to pass therethrough.

The foldable card may include a seventh panel hingedly connected to the sixth panel.

It is further disclosed that the first panel and the third panel may define the same length, the first panel and the fifth panel may define the same length, the first panel and the second panel may define the same length, and/or the first panel and the seventh panel may define the same length.

The foldable card may be made from at least one of cardboard, laminated paperboard, or plastic.

Additionally, it is disclosed that the first panel may define a third opening configured to allow a portion of a third surgical instrument to pass therethrough, the third panel may define a third opening configured to allow a portion of the third surgical instrument to pass therethrough.

The first opening of the first panel may define a non-circular shape.

The present disclosure also relates to a kit comprising a pouch, a first surgical instrument disposed within the pouch, a second surgical instrument disposed within the pouch, and a foldable card disposed within the pouch. The foldable card includes a first panel, a second panel, and a third panel. The first panel defines a first opening first opening configured to allow a portion of the first surgical instrument to pass therethrough, and a second opening configured to allow a portion of the second surgical instrument to pass therethrough. The second panel is hingedly connected to the first panel, and the third panel is hingedly connected to the second panel. The third panel defines a first opening configured to allow a portion of the first surgical instrument to pass therethrough, and a second opening configured to allow a portion of the second surgical instrument to pass therethrough.

The pouch may be made of high density polyethylene fibers.

It is further disclosed that the first surgical instrument may be a cannula, the second surgical instrument may be an obturator, and the third surgical instrument may be a syringe.

The kit may also include a third surgical instrument disposed within the pouch. The first panel of the foldable card may define a third opening configured to allow a portion of the third surgical instrument to pass therethrough, and the third panel of the foldable card may define a third opening configured to allow a portion of the third surgical instrument to pass therethrough.

Additionally, it is disclosed that the first opening of the third panel of the foldable card may define a non-circular shape. It is further disclosed that the third panel of the foldable card may extend from the fourth panel of the foldable card at a non-perpendicular angle when the first surgical instrument passes through the first opening of the first panel of the foldable card and through the first opening of the third panel of the foldable card.

In aspects, the foldable card may also include a fourth panel hingedly connected to the third panel, a fifth panel hingedly connected to the fourth panel, and a sixth panel hingedly connected to the fifth panel.

### BRIEF DESCRIPTION OF THE DRAWINGS

Various aspects and features of the present disclosure are described herein with reference to the drawings, which are incorporated in and constitute a part of this specification, and together with a general description of the disclosure given above and the detailed description below, serve to explain the principles of the disclosure, wherein:

FIG. 1 is a front view of a foldable card in an unfolded configuration, in accordance with the present disclosure;

FIG. 2 is a perspective view of the foldable card of FIG. 1 in a partially folded configuration;

FIG. 3 is a perspective view of the foldable card of FIGS. 1 and 2 in a fully folded configuration;

FIG. 4 is a side view of the foldable card of FIGS. 1-3 in a fully folded configuration and with surgical instruments therein; and

FIG. 5 is a perspective view of the foldable card of FIGS. 1-4 in a fully folded configuration with surgical instruments therein and within a package.

### DETAILED DESCRIPTION

Particular aspects of the present disclosure are described hereinbelow with reference to the accompanying drawings. However, it is to be understood that the disclosed aspects are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. Like reference numerals refer to similar or identical elements throughout the description of the figures.

Reference will now be made in detail to aspects of the present disclosure. While certain aspects of the present disclosure will be described, it will be understood that it is not intended to limit the present disclosure. To the contrary, reference to various aspects of the present disclosure is intended to cover alternatives, modifications, and equivalents as may be included within the spirit and scope of the present disclosure as defined by the appended claims.

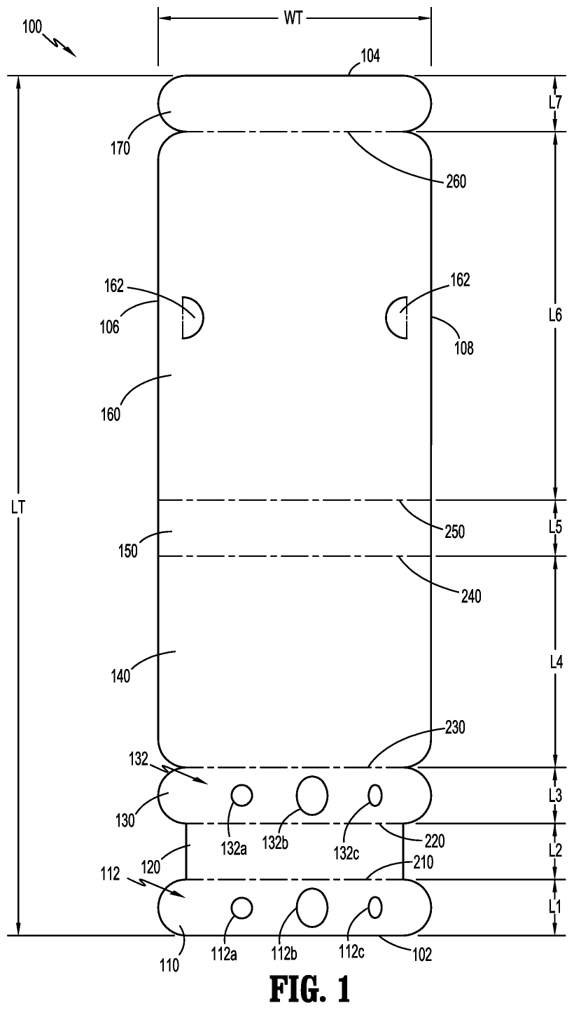

FIG. 1 illustrates a foldable card 100 in an unfolded configuration, in accordance with an aspect of the present disclosure. Generally, the foldable card 100 is configured to be folded in a manner to securely hold surgical instrumentation therein.

The foldable card 100 defines a first end 102, a second end 104, a first lateral side 106, and a second lateral side 108. The foldable card 100 includes a first panel 110, a second panel 120, a third panel 130, a fourth panel 140, a fifth panel 150, a sixth panel 160, and a seventh panel 170. Additionally, a first fold line 210 hingedly interconnects the first panel 110 and the second panel 120, a second fold line 220 hingedly interconnects the second panel 120 and the third panel 130, a third fold line 230 hingedly interconnects the third panel 130 and the fourth panel 140, a fourth fold line 240 hingedly interconnects the fourth panel 140 and the fifth panel 150, a fifth fold line 250 hingedly interconnects the fifth panel 150 and the sixth panel 160, and a sixth fold line 260 hingedly interconnects the sixth panel 160 and the seventh panel 170.

With continued reference to FIG. 1, the foldable panel 100 defines a total length “LT” and a total width “WT.” Each panel of the foldable card 100 defines a length: the first panel 110 defines a length “L1,” the second panel 120 defines a length “L2,” the third panel 130 defines a length “L3,” the fourth panel 140 defines a length “L4,” the fifth panel 150 defines a length “L5,” the sixth panel 160 defines a length “L6,” and the seventh panel 170 defines a length “L7.” Examples of these measurements are provided and are suited for surgical instruments 500 shown, however other measures are contemplated and may be determined in part by the particular surgical instrument(s) designed to be used therewith, for instance. It is envisioned that the total length “LT” of the foldable card 100 is between about 10 inches and about 20 inches (e.g., about 15 inches), and the total width “WT” of the foldable card 100 is between about 4.0 inches and about 5.5 inches (e.g., about 4.9 inches). It is also envisioned that “L1” is between about 0.5 inches and about 1.5 inches (e.g., about 1.0 inches), “L2” is between about 0.5 inches and about 1.5 inches (e.g., about 1.0 inches), “L3” is between about 0.5 inches and about 1.5 inches (e.g., about 1.0 inches), “L4” is between about 3.0 inches and about 4.0 inches (e.g., about 3.5 inches), “L5” is between about 0.5 inches and about 1.5 inches (e.g., about 1.0 inches), “L6” is between about 7.0 inches and about 8.5 inches (about 7.8 inches), and “L7” is between about 0.5 inches and about 1.5 inches (about 1.0 inches). Further, it is envisioned that any or all of “L1,” “L2,” “L3,” “L5,” and “L7” are the same length as each other or not the same length as each other. While the measurements listed herein is in no way limiting and is provided as an example, other measurements are clearly included in the scope of the present disclosure.

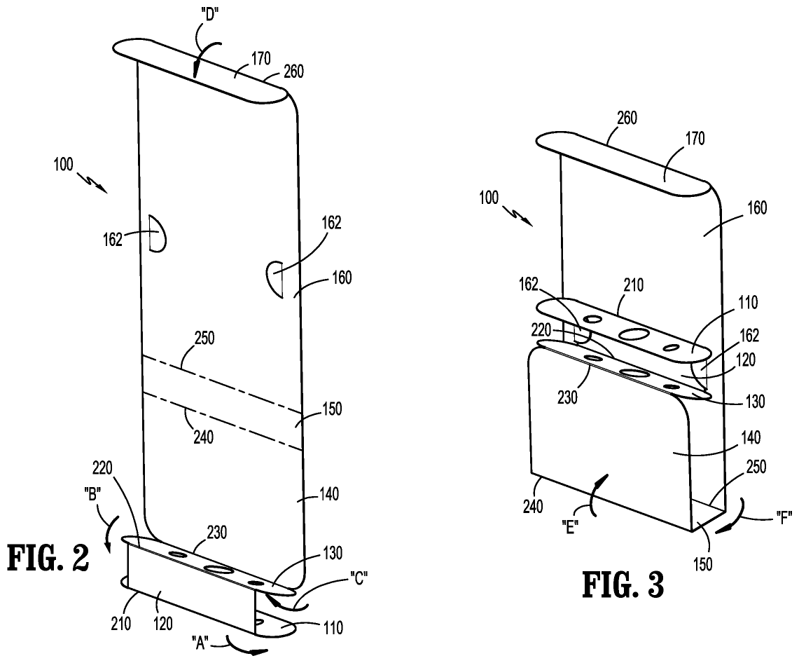

With particular reference to FIGS. 2 and 3, the foldable card 100 is shown in various stages of being folded. In FIG. 2, the foldable card 100 has been folded along the first fold line 210, the second fold line 220, and the sixth fold line 260. In particular, the first panel 110 has been folded in the general direction of arrow “A” (counter-clockwise, as shown in FIG. 2) relative to the second panel 210, the second panel 210 has been folded in the general direction of arrow “B” (counter-clockwise) relative to the third panel 130, the third panel 130 has been folded in the general direction of arrow “C” (clockwise) relative to the fourth panel 140, and the seventh panel 170 has been folded in the general direction of arrow “D” (counter-clockwise) relative to the sixth panel 160.

In FIG. 3, the foldable card 100 has been folded along the fourth fold line 240, and the fifth fold line 250. In particular, the fourth panel 140 has been folded in the general direction of arrow “E” (clockwise) relative to the fifth panel 150, and the fifth panel 150 has been folded in the general direction of arrow “F” (clockwise) relative to the sixth panel 160.

Additionally, in the illustrated aspect, the sixth panel 160 includes a pair of tabs 162. The tabs 162 are spaced from lateral edges of the sixth panel 160 and each tab 162 is configured to engage a side of the second panel 120 after the foldable card 100 has been folded into the orientation shown in FIG. 3. The engagement between the tabs 162 and the second panel 120 helps maintain the orientation of the foldable card 100 (e.g., in response to the removal of a surgical instrument 500 from the foldable card 100). Further, while two tabs 162 are shown, the sixth panel 160 may include one tab 162 or more than two tabs 162.

While the specification and figures describe and may depict a particular order of folding the various panels of the foldable card 100, the foldable card 100 is able to be folded in any order without departing from the scope of the present disclosure.

As shown in FIGS. 1-3, the first panel 110 defines a plurality of openings (i.e., one or more openings) 112 extending therethrough, and the third panel 130 defines a plurality of openings i.e., one or more openings) 132 extending therethrough. More particularly, the first panel 110 defines a first opening 112a, a second opening 112b, and third opening 112c, and the third panel 130 defines a first opening 132a, a second opening 132b, and a third opening 132c. With specific reference to FIG. 1, the first opening 112a of the first panel 110 is aligned with the first opening 132a of the third panel 130 along a first axis “a-a,” the second opening 112b of the first panel 110 is aligned with the second opening 132b of the third panel 130 along a second axis “b-b,” and the third opening 112c of the first panel 110 is aligned with the third opening 132c of the third panel 130 along a third axis “c-c.”

In disclosed aspects, the fifth panel 150 also includes at least one opening extending therethrough. Here, a distal portion of a surgical instrument that does not require protection (e.g. a blunt tip) can pass through. Instruments having a sharp tip would not extend through an opening in the fifth panel 150, for instance.

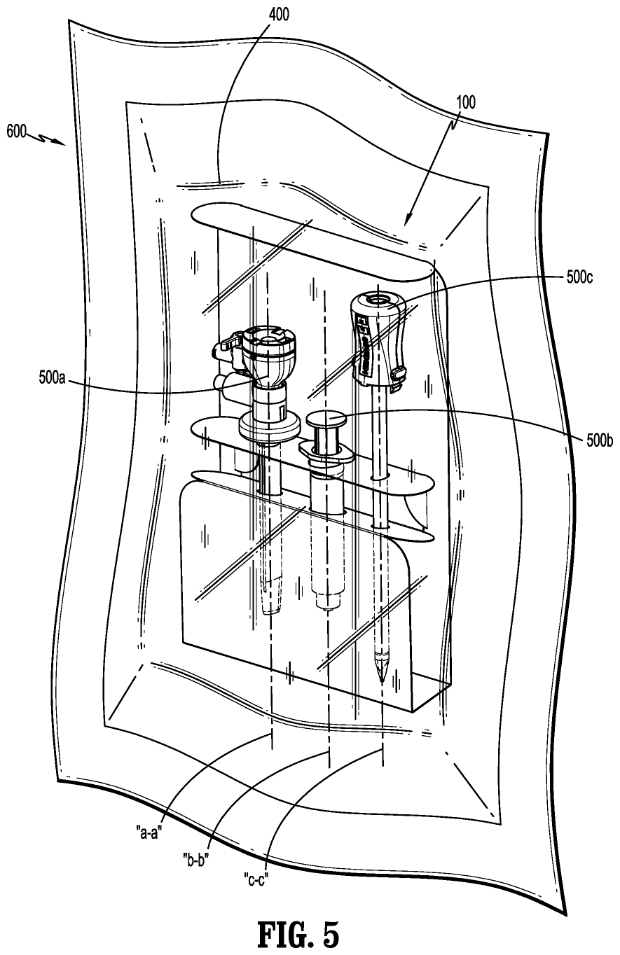

Referring now to FIGS. 1 and 5, the foldable card 100 is shown within a package or pouch 400 (e.g., entirely within), and with surgical instruments 500 held by the foldable card 100 and within (e.g., entirely within) the package or pouch 400. More specifically, a first surgical instrument 500a is shown disposed along the first axis “a-a” with portions thereof extending through the first opening 112a of the first panel 110, and through the first opening 132a of the third panel 130. A second surgical instrument 500b is disposed along the second axis “b-b” with portions thereof extending through the second opening 112b of the first panel 110, and through the second opening 132b of the third panel 130. Additionally, a third surgical instrument 500c is disposed along the third axis “c-c” with portions thereof extending through the third opening 112c of the first panel 110, and through the third opening 132c of the third panel 130. In aspects where the first panel 110 includes one opening 112, and the third panel 130 includes one opening 132, the foldable card 100 is configured to hold a single surgical instrument.

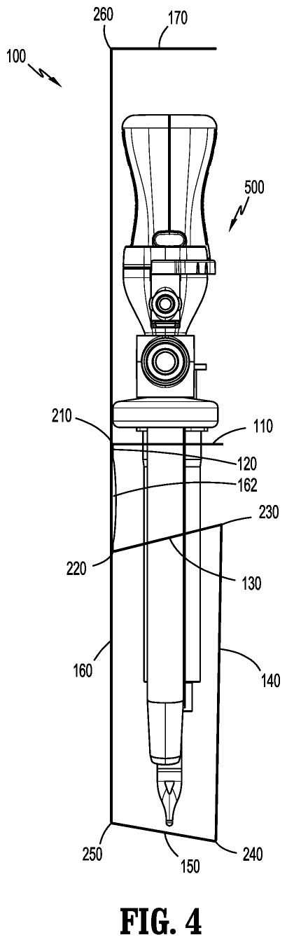

While each of the first panel 110 and the third panel 130 are shown with three openings 112, 132, respectively, it is envisioned that the first panel 110 and the third panel 130 define more or fewer than three openings to accommodate a corresponding number of surgical instruments 500. Additionally, while openings 112 and 132 of various shapes and sizes are shown, the present disclosure also includes aspects of the foldable card 100 having openings 112, 132 of sizes and shapes other than those shown, and in a different order than shown. For instance, the openings 112, 132 may be round (e.g., circular, non-circular, oval, elliptical, etc.), non-round (e.g., including straight lines and/or corners), or a combination thereof (e.g., eyelid-shaped), depending, for example, on the shape (e.g., cross-section) of relevant portions of a surgical instrument designed to be inserted therethrough. It is envisioned that non-circular openings 112, 132 may help secure circular portions (cross-sections) of surgical instruments that are inserted therethrough, especially, for instance, when the third panel 130 is disposed at a non-perpendicular angle with regard to the fourth panel 140 (see FIG. 4). Additionally, while the openings 112 of the first panel 110 are shown as the same size and shape as the corresponding openings 132 of the third panel 130 (e.g., the first opening 112a of the first panel 110 and the first opening 132a of the third panel 130), corresponding openings may be shaped and/or sized differently from each other depending on the size and shape of relevant portions of a surgical instrument designed to be inserted therethrough, for instance.

In various aspects, the fourth panel 140 of the foldable card 100 includes at least one viewing window, allowing portions of the surgical instruments 500 behind the fourth panel 140 to be seen by a user. For instance, such a viewing window may allow a user to view the shaft and/or distal tip of the surgical instrument 500 when the foldable card 100 and surgical instruments 500 are within the package 400.

In the example illustrated in FIG. 5, the pouch or package 400, the foldable card 100, and three surgical instruments 500 are shown. The first surgical instrument 500a is a cannula, the second surgical instrument 500b is a syringe, and the third surgical instrument 500c is an obturator. These three surgical instruments 500 are commonly used together in a single surgical procedure. Thus, using the package 400 and the foldable card 100 is a convenient way to package, ship, handle, and store surgical instruments used for a surgical procedure. While these particular surgical instruments are shown, the package 400 and the foldable card 110 can be configured to hold different types of surgical instruments, such as three different surgical instruments used during a single surgical procedure.

It is envisioned that the package 400 is made at least in part from a transparent or translucent material, such as high density polyethylene fibers (e.g., Tyvek®, sold by DuPont) to allow users to see the surgical instruments 500 within the package 400. Additionally, the package 400 creates a sterile barrier therein to help keep the surgical instruments 500 in a sterile environment during shipping and storage, for instance. Further, use of the foldable card 100 within the package 400 helps prevent the surgical instruments 500 from becoming loose during shipping, which thus helps prevent damaging the surgical instruments 500 and damaging the package 400.

The foldable card 100 may be made from cardboard, laminated paperboard, plastic (e.g., polyethylene terephthalate glycol (“PTEG”, polyethylene terephthalate (“PET”), polyethylene (“PE”), or other suitable materials.

Accordingly, the foldable card 100 is relatively low cost, fast and easy to assembly (i.e., folding the foldable card 100 along six fold lines), fast and easy to insert surgical instruments 500 through the openings 112, 132. The package 400 and foldable card 100 provides a convenient, secure, and sterile solution for shipping, handling and storing surgical instruments 500.

The present disclosure also includes a kit 600 (FIG. 5), which includes the foldable card 100, the package 400, and surgical instruments 500. Additionally, the present disclosure includes a method of packaging surgical instruments, including inserting portions of the surgical instruments 500 through openings 112, 132 of the foldable card 100, and wrapping the foldable card 100 and the surgical instruments 500 within a pouch or package 400.

It should be understood that the foregoing description is only illustrative of the disclosure. Various alternatives and modifications can be devised by those skilled in the art without departing from the disclosure. Accordingly, the disclosure is intended to embrace all such alternatives, modifications and variances. The aspects described with reference to the attached drawing figures are presented only to demonstrate certain examples of the disclosure. It is to be understood that the particular examples and aspects set forth herein are non-limiting, and modifications to structure, dimensions, materials, and methodologies may be made without departing from the scope of the present disclosure. Other elements, steps, methods and techniques that are insubstantially different from those described above and/or in the appended claims are also intended to be within the scope of the disclosure.

What is claimed:

1. A foldable card for retaining a plurality of surgical instruments, the foldable card comprising:
a first panel defining a first opening and a second opening, the first opening configured to allow a portion of a first surgical instrument to pass therethrough, the second opening configured to allow a portion of a second surgical instrument to pass therethrough;
a second panel hingedly connected to the first panel;
a third panel hingedly connected to the second panel and defining a first opening and a second opening, the first opening configured to allow a portion of the first surgical instrument to pass therethrough, the second opening configured to allow a portion of the second surgical instrument to pass therethrough;
a fourth panel hingedly connected to the third panel;
a fifth panel hingedly connected to the fourth panel; and
a sixth panel hingedly connected to the fifth panel.

2. The foldable card according to claim 1, further including a seventh panel hingedly connected to the sixth panel.

3. The foldable card according to claim 1, wherein the first panel and the third panel define the same length.

4. The foldable card according to claim 3, wherein the first panel and the fifth panel define the same length.

5. The foldable card according to claim 4, wherein the first panel and the second panel define the same length.

6. The foldable card according to claim 5, wherein the first panel and the seventh panel define the same length.

7. The foldable card according to claim 1, wherein the foldable card is made from at least one of cardboard, laminated paperboard, or plastic.

8. The foldable card according to claim 1, wherein the first panel defines a third opening configured to allow a portion of a third surgical instrument to pass therethrough.

9. The foldable card according to claim 8, wherein the third panel defines a third opening configured to allow a portion of the third surgical instrument to pass therethrough.

10. The foldable card according to claim 1, wherein the first opening of the first panel defines a non-circular shape.

11. A kit comprising:
a pouch;
a first surgical instrument disposed within the pouch;
a second surgical instrument disposed within the pouch; and
a foldable card disposed within the pouch, the foldable card including:
a first panel defining a first opening and a second opening, the first opening configured to allow a portion of the first surgical instrument to pass therethrough, the second opening configured to allow a portion of the second surgical instrument to pass therethrough;
a second panel hingedly connected to the first panel; and
a third panel hingedly connected to the second panel and defining a first opening and a second opening, the first opening configured to allow a portion of the first surgical instrument to pass therethrough, the second opening configured to allow a portion of the second surgical instrument to pass therethrough.

12. The kit according to claim 11, wherein the pouch is made of high density polyethylene fibers.

13. The kit according to claim 11, wherein the first surgical instrument is a cannula, and wherein the second surgical instrument is an obturator.

14. The kit according to claim 11, further including a third surgical instrument disposed within the pouch, wherein the first panel of the foldable card defines a third opening configured to allow a portion of the third surgical instrument to pass therethrough, and wherein the third panel of the foldable card defines a third opening configured to allow a portion of the third surgical instrument to pass therethrough.

15. The kit according to claim 14, wherein the first surgical instrument is a cannula, wherein the second surgical instrument is an obturator, and wherein the third surgical instrument is a syringe.

16. The kit according to claim 11, the first opening of the third panel of the foldable card defines a non-circular shape.

17. The kit according to claim 16, wherein the third panel of the foldable card extends from the fourth panel of the foldable card at a non-perpendicular angle when the first surgical instrument passes through the first opening of the first panel of the foldable card and through the first opening of the third panel of the foldable card.

18. The kit according to claim 16, wherein the foldable card further includes a fourth panel hingedly connected to the third panel, a fifth panel hingedly connected to the fourth panel, and a sixth panel hingedly connected to the fifth panel.
